(19) United States
(12) Patent Application Publication
(10) Pub. No.: US 2009/0111671 A1
(43) Pub. Date: Apr. 30, 2009

(54) Exercise device and method for testing and/or strengthening muscles of the pelvic diaphragm

(76) Inventors: Kristin Keller Campbell, Ogden, UT (US); Mark Kramer Campbell, Ogden, UT (US)

Correspondence Address: Ms. Kristin K. Campbell, 4515 South 850 East, Ogden, UT, 84403, US

(21) Appl. No.: 11/978,867

(22) Filed: Oct. 30, 2007

(51) Int. Cl. A63B 23/00; A61M 31/00

(52) U.S. Cl. 482/148; 604/509

(57) ABSTRACT

An adjustable, weight resistance exercise device and method for testing and/or strengthening muscles of the pelvic diaphragm comprising an elongated, semi-rigid tube and a resilient balloon member. The tube has a first end that is formed to accommodate a hose barb and a second end. The balloon member covers the second end approximately two inches to a sealed end. A syringe is connected to the hose barb allowing fluid or gas such as water or air to flow through a passageway of the tube into the balloon member. The balloon member expands under the pressure forming a reservoir of varying diameter, length, and weight.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND OF THE INVENTION—FIELD OF INVENTION

The present invention relates to exercise devices and methods used for testing and/or strengthening the muscles of the pelvic diaphragm. More specifically, the present invention relates to an adjustable, weight resistance exercise device and method used to increase the muscle mass, strength, and tone of the pelvic floor and sphincter muscles.

### BACKGROUND OF THE INVENTION

The pelvic diaphragm consists primarily of two separate and distinct muscle groups, the levator ani muscle, a broad, thin muscle situated on the side of the pelvis, and the coccygeus muscle, a triangular plane of muscular and tendinous fibers situated dorsal to the levator ani. The levator ani muscle can be subdivided into the pubococcygeus or pelvic floor muscles and the sphincter vaginae or sphincter muscles. The pelvic floor muscles are elongated strands of muscle extending between the inner regions of the pelvic bone, supporting the perimeter of the vagina. This particular muscle configuration is commonly referred to in the medical community as having the appearance of hammocks having the ends attached to the pelvic bone inner perimeter while the middle portion of the hammocks overlays the exterior perimeter of the vagina. The pelvic floor and sphincter muscles operate in conjunction to constrict the opening and closing of the urethra, vagina, and rectum. When the pelvic diaphragm muscle tone is good, these openings are properly maintained and constricted by virtue of the tightness of the muscles surrounding the respective openings. However, numerous factors cause or significantly contribute to the deterioration of the pelvic diaphragm muscle structure and the subsequent enlarging and relaxing of the aforementioned openings. The primary factors contributing to weakened pelvic diaphragm muscle structure are childbirth, over medicating, poor physical conditioning, auto accidents, surgical procedures, progressive illnesses, and atrophy associated with aging.

The deterioration of the pelvic diaphragm muscle structure contributes to a number of medical conditions including uterine prolapse, fallen bladder, fallen rectum, cystitis, difficulties with voiding, decreased sexual comfort and performance, chronic vaginal and lower back discomfort, and various forms of incontinence. Many of these medical conditions are curable only through heavy medication or costly and painful surgery; however, proper pelvic diaphragm muscle tone has been shown to significantly reduce the occurrences of many of the above mentioned medical conditions. Although simply maintaining proper pelvic diaphragm muscle tone is an effective method for reducing numerous medical conditions associated with the deterioration of these muscles, it appears that the public is unwilling to utilize the current devices and methods for strengthening these particular muscles. This is unfortunate as 10 to 35% of female adults suffer from various forms of medical conditions associated with pelvic diaphragm muscle deterioration. Incontinence, in particular, plagues over 13 million Americans and over one-half of the nursing home residents in the United States. These numbers clearly represent the need for a simple device used to exercise and strengthen muscles of the pelvic diaphragm.

Various intravaginal devices for exercising and strengthening the pelvic floor muscles have been developed. A number of devices, known as Perineometers, were invented by Dr. Arnold Kegel. These devices use biofeedback as a diagnostic tool and as a tool for enabling a woman to initially learn voluntary muscle control. However, these devices do not provide suitable resistance to contraction of vaginal muscles to be suitable in a rigorous program of muscle rebuilding.

U.S. Pat. No. 2,507,858 and U.S. Pat. No. 2,541,520 have a pressurized sleeve with a rigid core and a tube that disconnects from the applicator. The expansible member is connected by a circular rim which fits between the flanges and clamps the inner free ends to create a fluid-tight relation. The pressurized sleeve is inserted into the vagina with a portion remaining outside of the body and inflated which is used to indicate pneumatically the change in pressure on an associated dial gage.

Another device for exercising the pelvic floor muscles is described in U.S. Pat. No. 3.752,150 which has an insert that extends outside of the vagina when inflated. A second tubing extends along the outer surface of the original tubing and passes through an opening which connects the other end of the tubular element and is in communication with the liquid supply and pump. The compressed air or other fluids produce and indication on a pressure gauge of the force of the pelvic floor muscle contractions.

U.S. Pat. No. 4,050,449 has a balloon that terminates at its outer second end in the tube which is connected by a collar. The tube is attached to a bifurcated tube which has a first fork that communicates to a pressure valve and a second fork that communicates to a syringe.

Also, U.S. Pat. No. 4,768,522 has an elongated rod that extends into a cuff portion that is made of a sponge-like resilient material having a multitude of interstices therein. The rod has a tubular body member that extends longitudinally from the free end portion which has a reinforcing means and a strap means with a plug to the inner end. The tubular body member has a reinforcing means or spring member positioned in the passageway. An air flow measuring means may be used for measuring the contractions of the pelvic floor muscles.

Limitations and disadvantages of the prior art are the relatively large size of the vaginal probes which cause some women discomfort or even pain during the process of insertion. Many women, especially older women, are reluctant to self-insert such a large, hard, and unyielding vaginal probe into their vaginas. Furthermore, these large probes protrude outside of the body cavity and stretch the sphincter muscles causing more damage and does not allow these muscles to be fully exercised.

These devices are not portable and require patients to make special arrangements and scheduling to complete the exercises; thus, making it impossible to be worn and used in private or public for extended periods of time. Furthermore, multiple parts of these devices make disassembly, cleaning, and reassembly a complex task.

The perineometers have a form of a large balloon member that is inserted into the vagina leaving a portion outside of the body which stretches the sphincter muscles and is uncomfortable to use. The present invention does not stretch the sphincter muscles because the balloon member is deflated and small when inserting and removing from the vagina and rectum which is also less intimidating to the user.

Furthermore, the perineometers are attached to a variety of measuring meters that are expensive and require a visit to a clinic or hospital to use. The present invention does not have a meter and tests the strength of the sphincter and pelvic floor muscles by gradually increasing the weight of the exercise device to find the correct weight in which the muscles can voluntarily hold the exercise device.

Additional structural advantages of the present invention are the adjustability of the balloon member to a varying size and weight to accommodate any size levator gap, referring to the relative gap between the sphincter muscles of the vagina, and muscle strength using gradual weight resistance exercising, a flexible and simple balloon member which conforms to the natural structure of each individual user, and the ability to be worn in public due to its light-weight and portability.

Another form of exercising the pelvic floor muscles is through weight resistance training. The principle of using vaginal weights for exercising is to facilitate the muscular tissue for contraction via sensory input from the contact of the weight with the muscular tissue.

Several vaginal weights have been invented such as U.S. Pat. No. 5,213,557, which discloses a device for exercising pelvic floor muscles having a set of two or more weights that fit together to form an elongated conical shape, and U.S. Pat. No. 5,407,412, wherein a set of devices of identical size and shape, but of different weight, are inserted into the vagina and, if the pelvic floor muscles are capable of retaining that weight, the device is replaced by a heavier device of the set. Furthermore, U.S. Pat. No. 5,554,092 describes a device with a singular hollow body designed to receive various configurations of weights.

Limitations and disadvantages of the prior art include the geometrical shape of the weights, as several of these have a conical shape at one or both ends. The shape may cause the weight, after insertion and during contraction of the musculature, to slide further up into the vagina and tilt so that its position becomes horizontal and, consequently, is placed so high that the exercise has no effect. Another problem with the conical shape is that the vaginal weight might slide out of the vagina.

Also, existing vaginal weights have the drawback that the weights are dimensioned so that the diameter and weight are proportional, which means that a larger diameter results in greater weight. Many users require different sizes of devices in order to provide a better fit for the exercise device. Studies have shown that up to 17% of a group of women could not use common vaginal weights because the size of the levator gap made tissue contact impossible. Thus, the problem is that a wide levator gap generally is a symptom of weak muscles, for which reason the patients cannot retain the weight. Furthermore, existing vaginal weights have the drawback of being heavy, thus, making them inconvenient to carry and use in public.

Additional patents were issued for a number of isometric exercisers, including U.S. Pat. No. 4,241,912 and U.S. Pat. No. 6,394,939, which both utilize a rounded shaft having a flange and handle attached to one end for insertion within the vagina leaving a portion of the handle protruding from the vagina, such that the device can be manipulated by the user. Exercise is accomplished through the movement of the pelvic floor muscles up and down on the rounded end of the device. Also, U.S. Pat. No. 6,224,525 comprising a pair of longitudinally extending members that are connected to one another at their first ends and opposing second ends of these members are movable between an open position and a closed position.

Limitations and disadvantages of the prior art include the discomfort of insertion of these devices within the vagina due to their hard and unyielding design, which must be inserted at their full size as they cannot be deflated before insertion. This may cause a pinching of the sensitive area inside the vagina. Furthermore, the user must grip the device with their hand to assist the exercise process.

Aside from vaginal exercise inserts, physicians have attempted to utilize medication in an attempt to cure the problems associated with poor pelvic floor muscle strength. However, there are specific disadvantages associated with medication type cures. These types of cures typically do not increase the strength of the relevant muscles, they only temporarily relieve the resulting symptoms associated with poor muscle strength. Medications for incontinence, for example, often offer only temporary relief to the patient until muscle strength can be restored through exercise. Incontinence medications, in addition to being only a temporary cure, can also further the symptoms by medically causing the muscles to further relax and, thus, become weak.

Therefore, although numerous methods and devices currently exist for exercising the pelvic floor muscles, the methods and devices currently used are seldom used properly or adhered to for a time period significant enough to benefit the patient. This is likely due to the physical shortcomings of the above mentioned devices, in addition to the inherent psychological objections involved with using any vaginally inserted device. Some devices are constructed and arranged as to create danger of injury to the pelvic floor muscles during insertion and/or during the exercising process. The size, complexity, and methods of use of the devices currently in the art are certain to increase these objections, thus reducing the effectiveness of the devices.

Other shortcomings of the current method and devices are that they cannot be adjusted to accommodate different combinations of sizes and weights. Additionally, the complexities involved with cleaning, transporting, and general use of these devices also hinder the effectiveness of the methods and devices.

### BRIEF SUMMARY OF THE INVENTION

The present invention relates to an adjustable, weight resistance exercise device and method used to increase the muscle mass, strength, and tone of the pelvic diaphragm muscle structure; thereby, preventing the need for medication or surgery due to illnesses related to weakened pelvic floor and sphincter muscles.

One preferred embodiment comprises an elongated, semi-rigid tube and a resilient balloon member. The tube has a first end that is formed to accommodate a hose barb and a second end. The balloon member covers the second end approximately two inches to a sealed end. A syringe is connected to the hose barb allowing fluid or gas such as water or air to flow through a passageway of the tube into the balloon member. The balloon member expands under the pressure forming a reservoir of varying diameter, length, and weight.

### BRIEF SUMMARY OF THE INVENTION—OBJECTS AND ADVANTAGES

Accordingly, several objects and advantages of the present invention are:
a) to provide an exercise device and method which increases the muscle mass, strength, and tone of the muscles of the pelvic diaphragm thereby constricting the opening and closing of the urethra, vagina, and rectum;
b) to provide an exercise device which reduces the need for medication or surgery for illnesses related to weakened muscles of the pelvic diaphragm;
c) to provide an exercise device which uses graduated weight resistance exercising;
d) to provide an exercise device which can be adjusted to accommodate a variety of diameter, length, and weight combinations;
e) to provide an exercise device which can be adjusted to accommodate different size levator gaps and pelvic diaphragm muscle strength;
f) to provide an exercise device which is deflated and small when inserting and removing from the vagina or rectum, thereby allowing muscles of the pelvic diaphragm to maintain their current strength;
g) to provide an exercise device which is easily and painlessly inserted and removed from the vagina and rectum;
h) to provide an exercise device which rests inside the vaginal cavity when inflated to better exercise the muscles of the pelvic diaphragm;
i) to provide an exercise device which does not further damage, stretch, or re-injure muscles of the pelvic diaphragm when inserting or removing from the vagina or rectum;
j) to provide an exercise device which is flexible and pliable, thereby conforming to the natural structure of each individual user;
k) to provide an exercise device which overcomes common psychologically objections for the user to use;
l) to provide an exercise device which can be used during the exercise process without the assistance of the user's hands;
m) to provide an exercise device which is light-weight and portable;
n) to provide an exercise device which can be used and worn in private or public for any proper period of time;
o) to provide an exercise device which stays more securely in the user's vagina or rectum due to the cylindrical shape;
p) to provide an exercise device which is round or cylindrical in geometric shape to prevent slippage;
q) to provide an exercise device which is comfortable and convenient to use;
r) to provide an exercise device which is simple to use and operate;
s) to provide an exercise device which is easy to inflate and deflate;
t) to provide an exercise device which is easy to clean and maintain as no assembly or disassembly is required.

These objects are accomplished through a reasonably sized and weighted elongated tube with inflation capabilities. Further objects and advantages of our invention will become apparent from a consideration of the drawings and ensuing description.

### BRIEF DESCRIPTION OF THE DRAWINGS

For a fuller understanding of the nature and objects of the invention, reference should be had to the following detailed description taken in connection with the accompanying drawings, in which:

FIG. 1 is a front view of a preferred embodiment of the exercise device of this invention.

FIG. 2 is a cross-sectional view of the invention of FIG. 1, taken along section line 2-2.

FIG. 3 is a cross-sectional view of the invention of FIG. 1, taken along section line 3-3.

FIG. 4 is an enlarged view of the invention of FIG. 3, encompassing the dashed circle 4.

FIG. 5 is a front view of the invention of FIG. 1, illustrating full inflation of the balloon member.

FIG. 6 is a cross-sectional view of the invention of FIG. 5, taken along section line 6-6.

FIG. 7 is a cross-sectional view of the invention of FIG. 5, taken along section line 7-7.

FIG. 8A is a cross-sectional view of the pelvic portion of the female human anatomy.

FIG. 8B is a cross-sectional view of the female human anatomy, illustrating the positioning of the deflated exercise device inserted within the vaginal cavity with the pelvic floor and sphincter muscles at rest.

FIG. 8C is a cross-sectional view of the female human anatomy, illustrating the positioning of the partially inflated exercise device inserted within the vaginal cavity with the pelvic floor and sphincter muscles contracted.

FIG. 8D is a cross-sectional view of the female human anatomy, illustrating the positioning of the fully inflated exercise device inserted within the vaginal cavity with the pelvic floor and sphincter muscles contracted.

FIG. 9A is a cross-sectional view of the pelvic portion of the male human anatomy.

FIG. 9B is a cross-sectional view of the male human anatomy, illustrating the positioning of the deflated exercise device inserted within the rectal cavity with the pelvic floor and sphincter muscles at rest.

FIG. 9C is a cross-sectional view of the male human anatomy, illustrating the positioning of the partially inflated exercise device inserted within the rectal cavity with the pelvic floor and sphincter muscles contracted.

FIG. 9D is a cross-sectional view of the male human anatomy, illustrating the positioning of the fully inflated exercise device inserted within the rectal cavity with the pelvic floor and sphincter muscles contracted.

FIG. 10 is a front view of the second embodiment of the invention.

FIG. 11 is a cross-sectional view of the invention of FIG. 10, taken along section line 11-11.

FIG. 12 is a front view of the invention of FIG. 10, illustrating full inflation of the balloon member.

FIG. 13 is a cross-sectional view of the invention of FIG. 12, taken along section line 13-13.

FIG. 14 is a cross-sectional view of the invention of FIG. 12, taken along section line 14-14.

FIG. 15 is a front view of the third embodiment of the invention.

FIG. 16 is a cross-sectional view of the invention of FIG. 15, taken along section line 16-16.

FIG. 17 is a front view of the invention of FIG. 15, illustrating full inflation of the reservoir.

FIG. 18 is a cross-sectional view of the invention of FIG. 17, taken along section line 18-18.

FIG. 19 is a cross-sectional view of the invention of FIG. 17, taken along section line 19-19.

FIG. 20 is a front view of the fourth embodiment of the invention.

FIG. 21 is a cross-sectional view of the invention of FIG. 20, taken along section line 21-21.

FIG. 22 is a front view of the invention of FIG. 20, illustrating full inflation of the reservoir.

FIG. 23 is a cross-sectional view of the invention of FIG. 22, taken along section line 23-23.

FIG. 24 is a cross-sectional view of the invention of FIG. 22, taken along section line 24-24.

FIG. 25 is a front view of the fifth embodiment of the invention.

FIG. 26 is a cross-sectional view of the invention of FIG. 25, taken along section line 26-26.

FIG. 27 is a front view of the invention of FIG. 25, illustrating full inflation of the expandable cover.

FIG. 28 is a cross-sectional view of the invention of FIG. 27, taken along section line 28-28.

FIG. 29 is a cross-sectional view of the invention of FIG. 27, taken along section line 29-29.

FIG. 30 is a front view of the sixth embodiment of the invention.

FIG. 31 is a front view of the invention of FIG. 30, illustrating full inflation of the balloon member.

FIG. 32 is a front view of an oval-shaped graduated weight set.

FIG. 33 is a front view of a barrel-shaped graduated weight set.

### DRAWINGS—REFERENCE NUMERALS

110, 210, 310, 410, 510, 610 exercise device
111, 211, 311, 411, 511 tube
112, 212, 312, 412, 512 first end
113, 213, 513 second end
114 tube taper
115, 315, 415, 515 passageway
116, 216 balloon member
117 sealed end
118, 418 bevel
119, 219, 419, 519 seal
120, 220, 320, 420, 520 reservoir
121, 321, 421 tip end
122, 322, 422 tip taper.
123 pelvic floor muscles
124 sphincter muscles
125 vagina
126 vaginal orifice
127 vaginal pelvic floor muscle platform
128 vaginal cavity
129 vaginal cavity edge
130 rectum
131 rectal orifice
132 rectal pelvic floor muscle platform
133 rectal cavity
134 rectal cavity edge
135 syringe
136, 636 hose barb
137 clip
138, 638 cap
339 reservoir edge
440 non-expandable cover
441, 541 first cover end
442, 542 second cover end
543 hole
544 expandable cover
545 second seal
546 plug
647 chain
648 first chain end
649 second chain end
650 hook
651A-E oval-shaped graduated weight set
652A-E barrel-shaped graduated weight set

### DETAILED DESCRIPTION OF THE INVENTION—PREFERRED EMBODIMENT

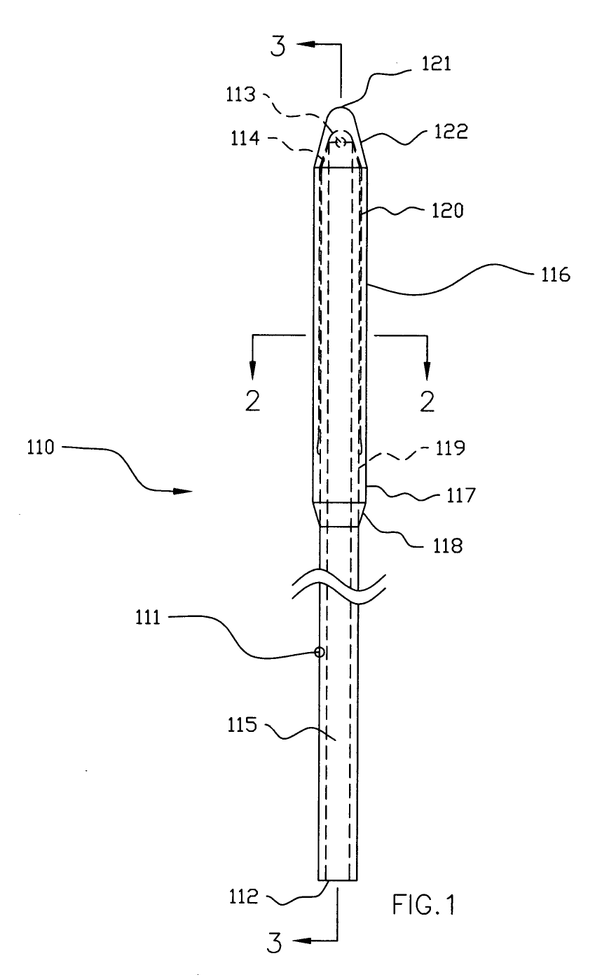

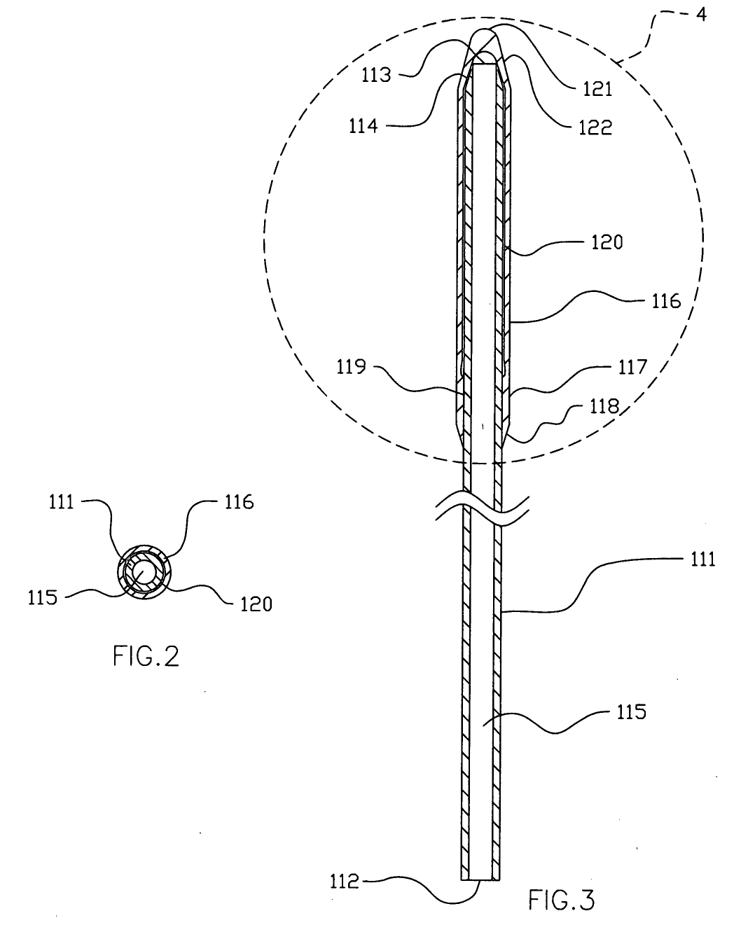

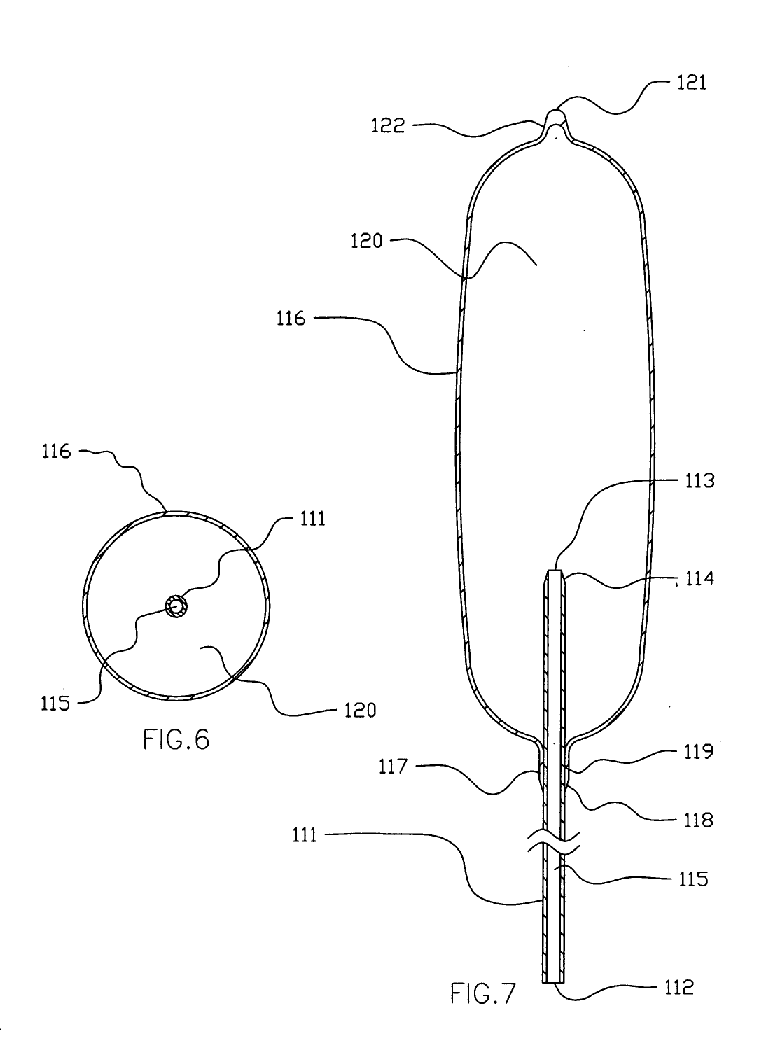

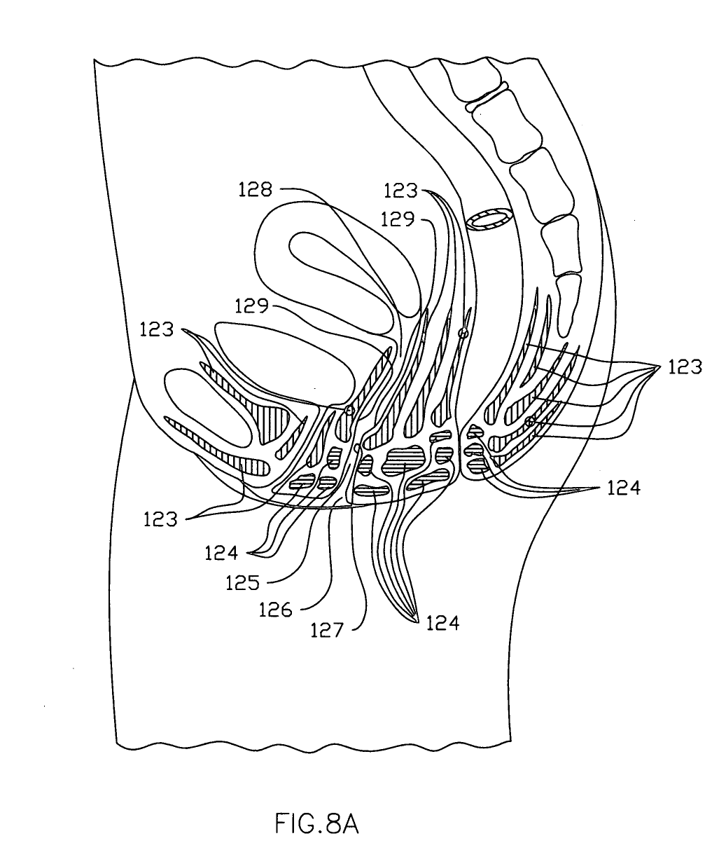

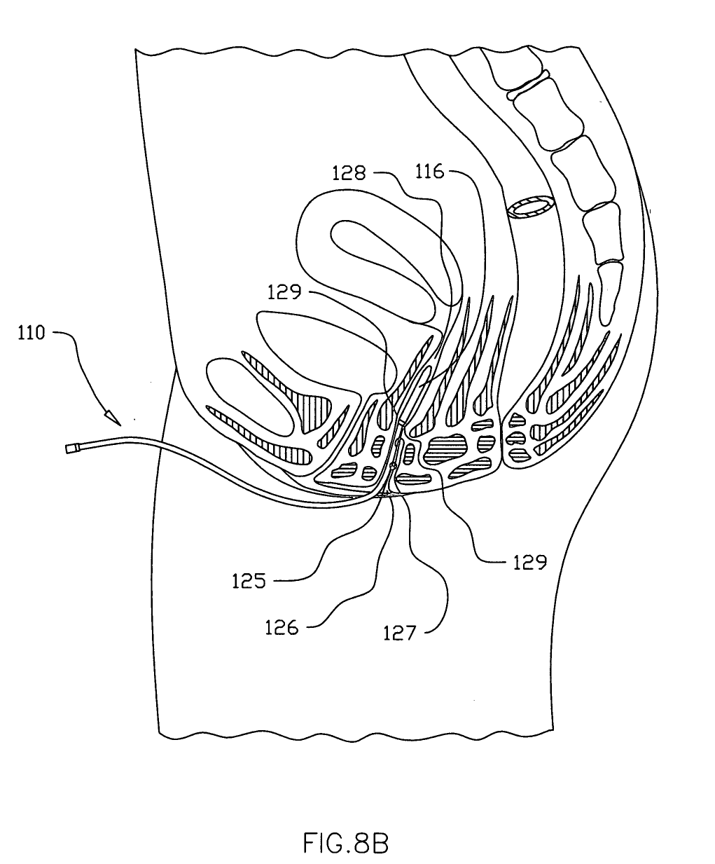

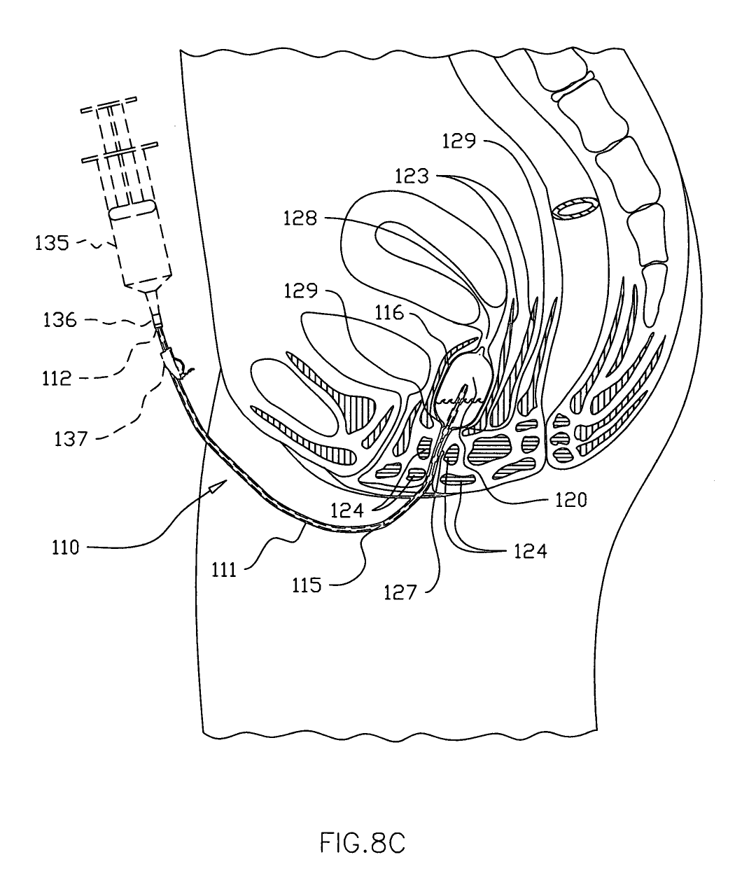

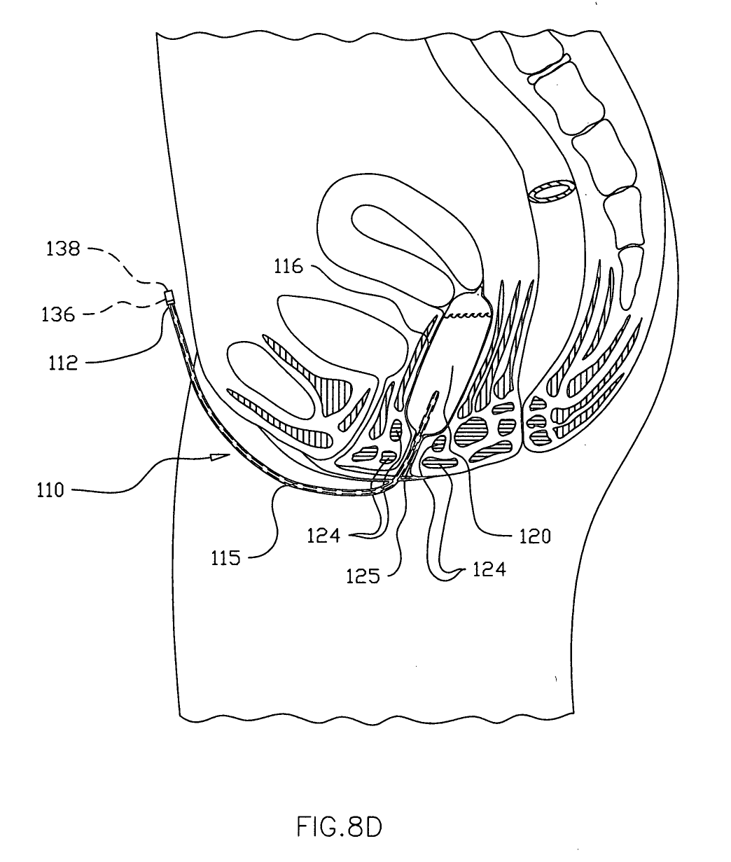

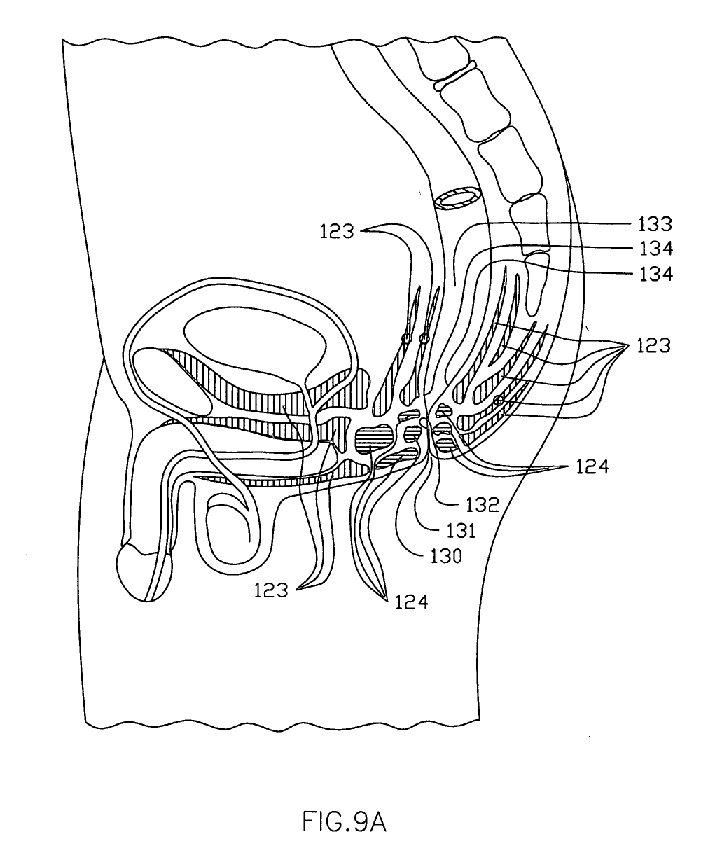

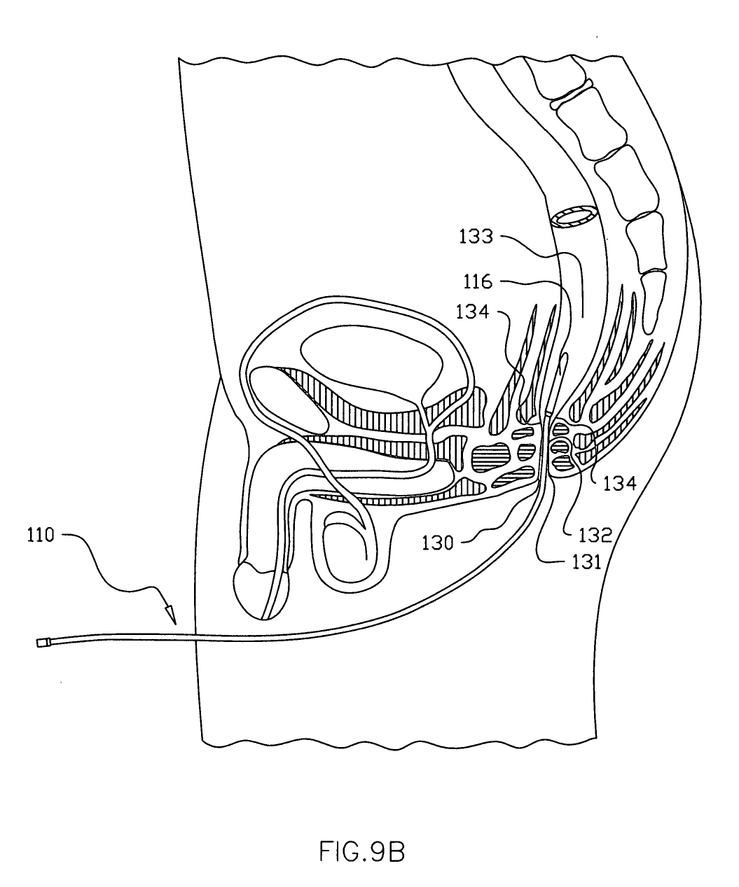

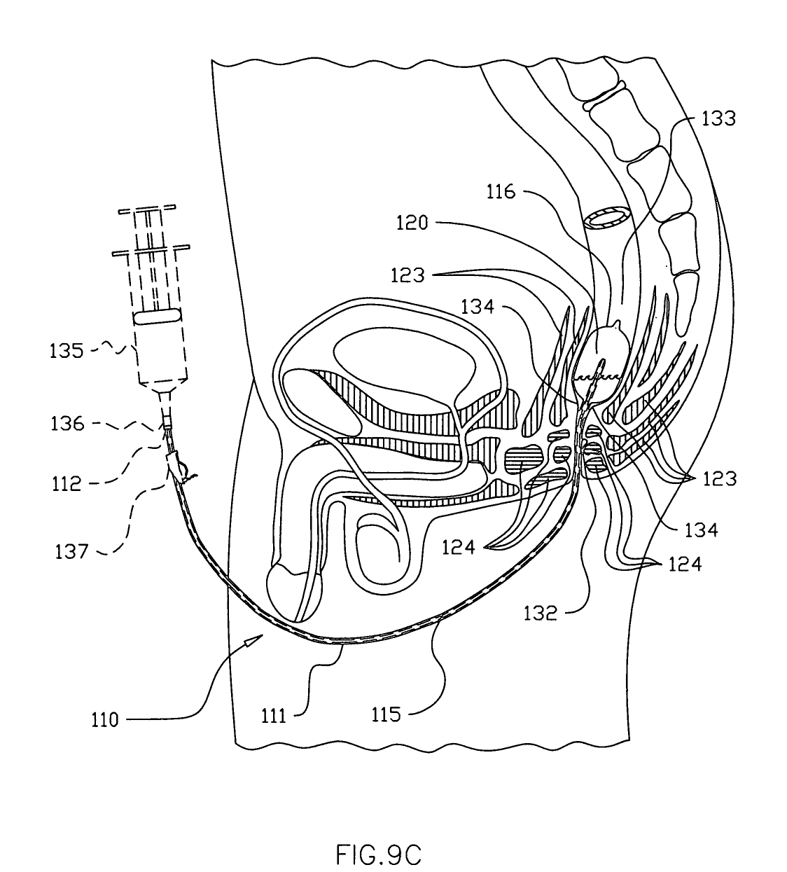

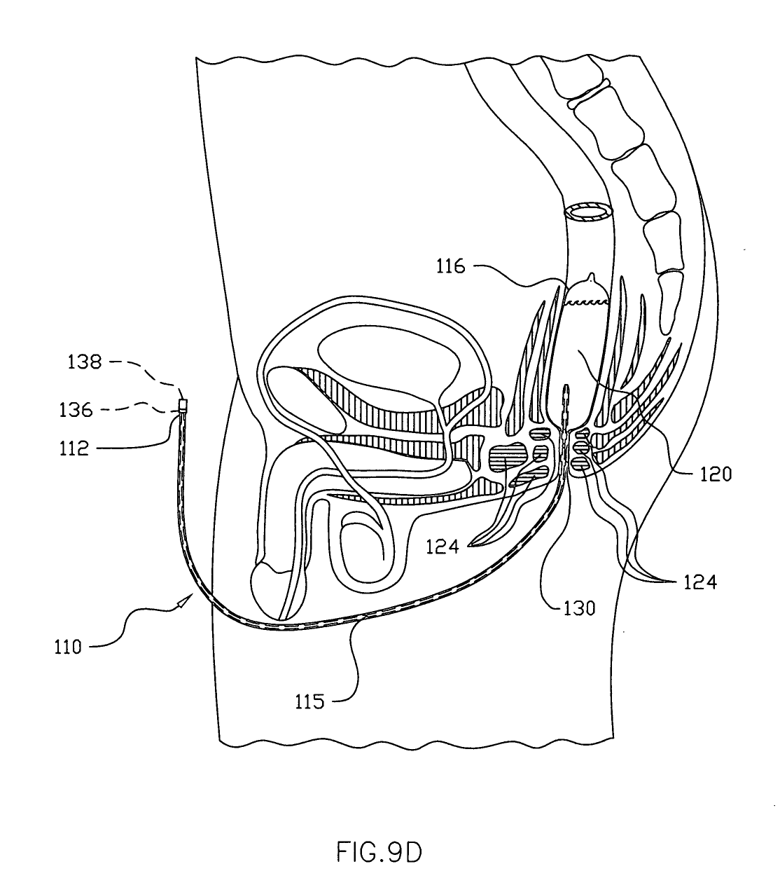

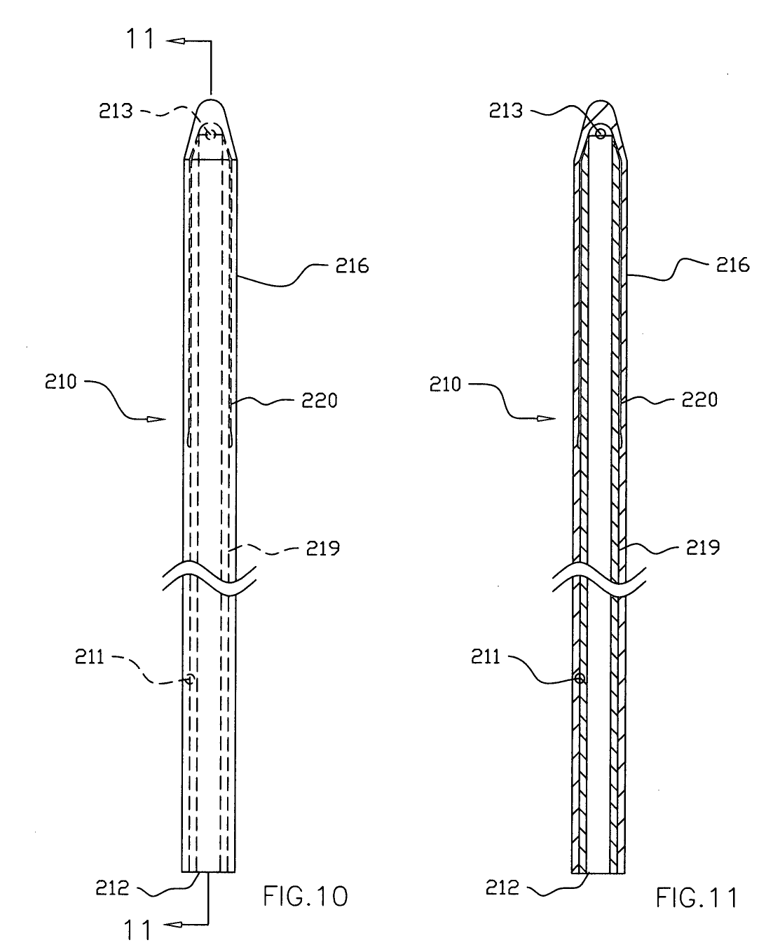

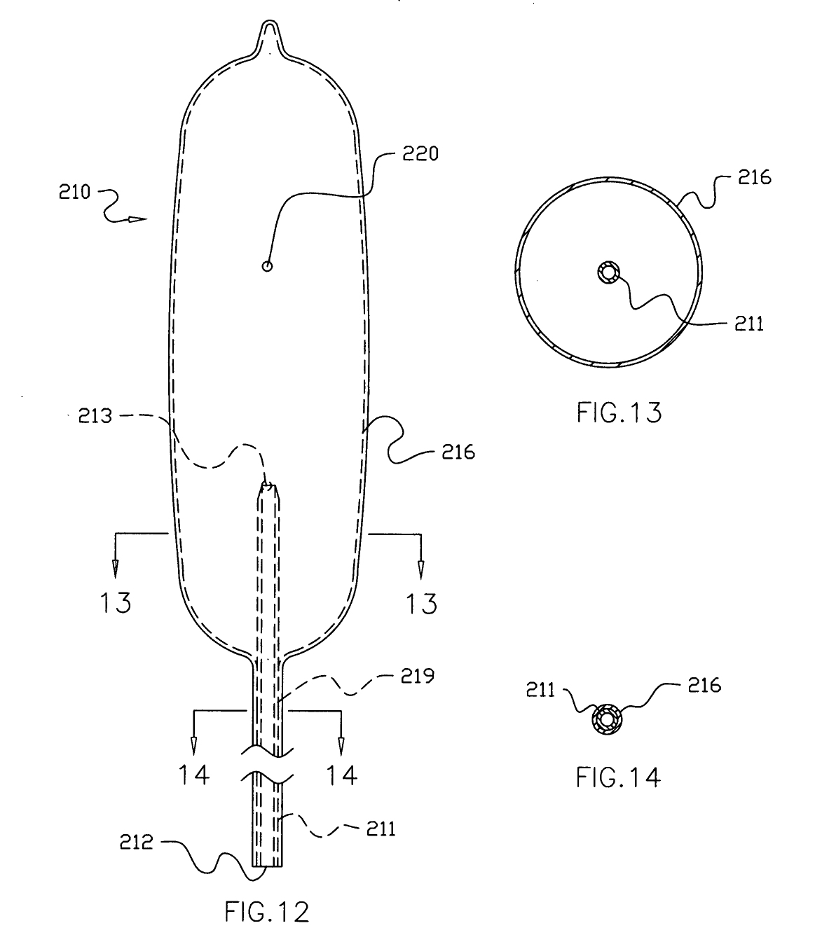

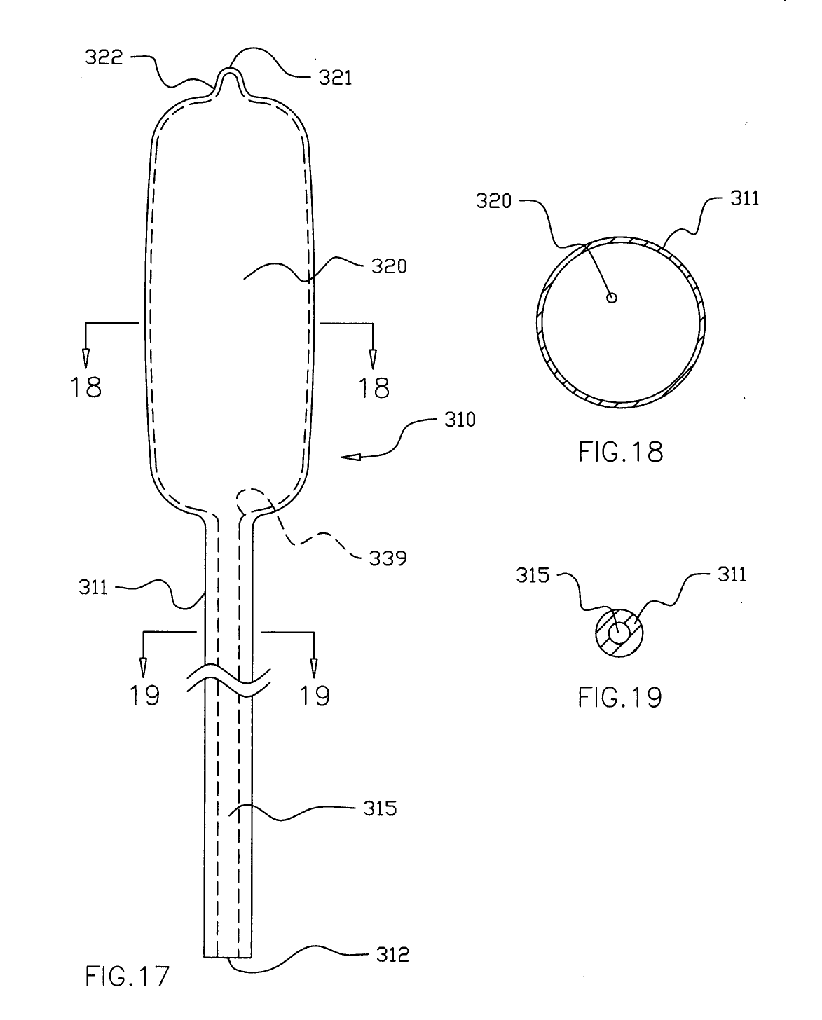

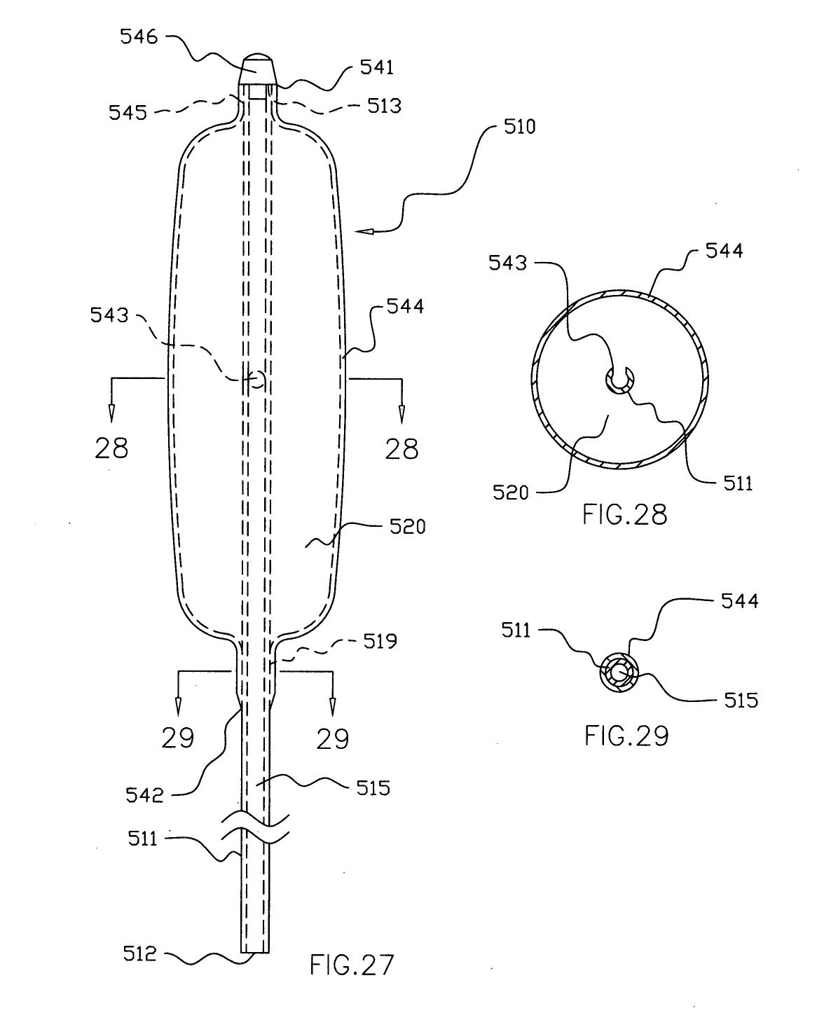

A preferred embodiment of the exercise device of this invention is illustrated in the drawing FIGS. 1-7, and the use of the exercise device is illustrated in FIGS. 8A-9D. Additional embodiments of the invention are shown in drawing FIGS. 10-33. An exercise device is generally indicated as 110 throughout FIGS. 1-9D and reference characters for similar parts for additional embodiments are increased in increments of 100. Referring first to the view of FIG. 1, it can be seen that exercise device 110 comprises, in general, a fill/evacuate hose, or tube 111 and a resilient covering, flexible bag, or balloon member 116.

As seen in FIGS. 1 and 3, tube 111 is a tubular-shaped hose that is approximately 3.0 to 16.0 inches in length, 0.25 inches in outer diameter, and 0.125 inches in inner diameter, but the length and diameters may be varied as long as comfort and ease of use is maintained, having a first end 112 and a second end 113, thereby extending into the interior of balloon member 116 from second end 113 to a sealed end 117. First end 112 is formed to accommodate a standard tube connecting device or hose barb 136, as shown in FIG. 8C, commercially manufactured in the industry. Second end 113 has a tube taper 114 that is approximately 35 degrees and 0.25 inches in length, but the angle and length may be varied as long as comfort and ease of use is maintained. An elongated channel or passageway 115 extends longitudinally through tube 111 from first end 112 to second end 113. Tube 111 is, generally, semi-rigid and flexible in construction, and may be made from materials such as medical grade rubbers, silicone, polyurethanes, or other suitable materials in any combination thereof, to permit insertion of exercise device 110 into a vagina 125, as shown in FIG. 8B, or a rectum 130, as shown in FIG. 9B.

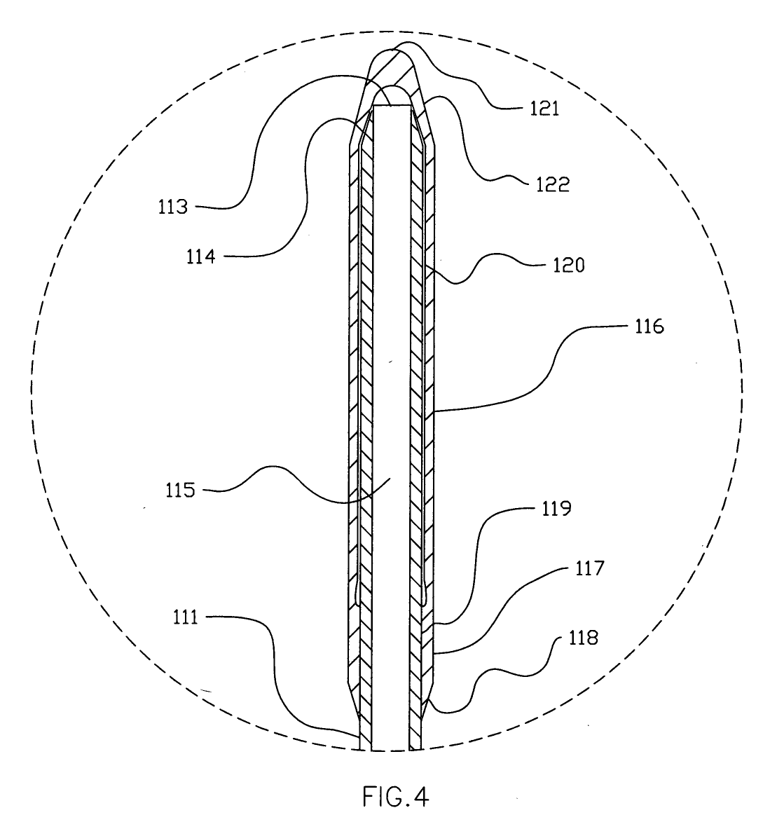

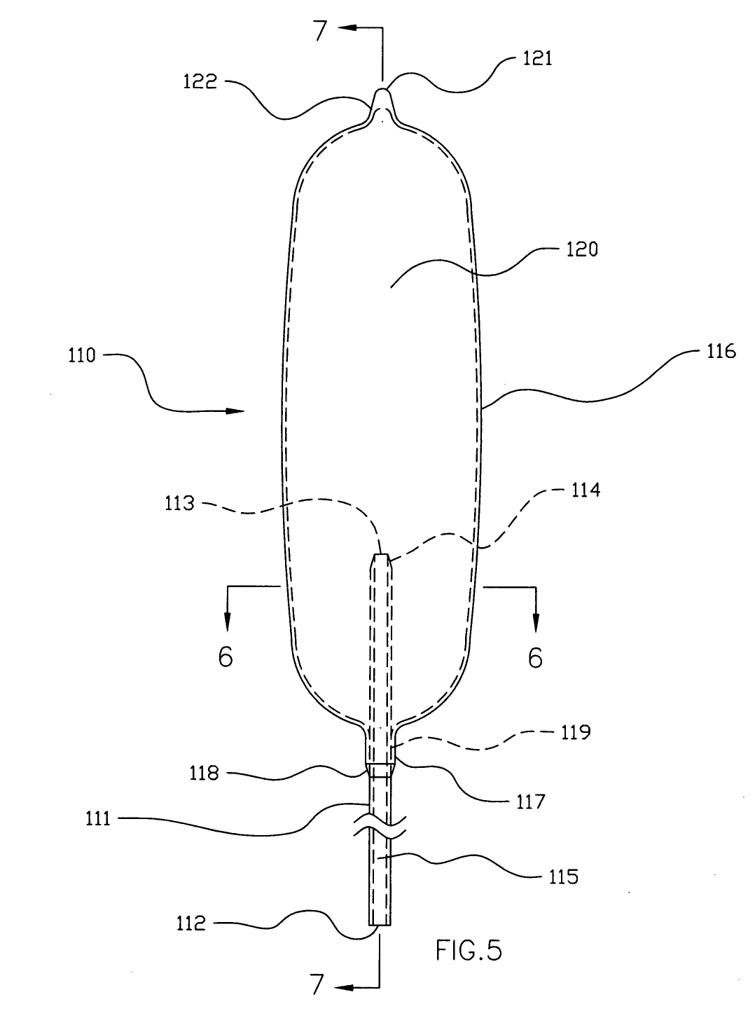

As seen in FIG. 4, balloon member 116 is a resilient cylindrical-shaped cap or flexible balloon that is approximately 0.5 to 3.0 inches in length, 0.3125 inches in outer diameter, and 0.25 inches in inner diameter, but the length and diameters may be varied as long as comfort and ease of use is maintained, that covers tube 111 from second end 113 to sealed end 117, having a sharp-angled cut or bevel 118 around the outer edge. Balloon member 116 is joined to tube 111 by a seal 119 that is approximately 0.25 to 0.5 inches in length, using a variety of known bonding techniques in the industry, forming a gap, chamber, or reservoir 120. As seen most clearly in FIG. 5, reservoir 120 expands cylindrically to accommodate a range that is approximately 0.5 to 7.0 inches in length and 0.3125 to 2.5 inches in diameter, but the length and diameter may be varied as long as comfort and ease of use is maintained. As seen in FIG. 4, balloon member 116 has a pointed end or tip end 121 that may vary in thickness due to fabrication techniques such as dip molded, injection molded, or extruded. Tip end 121 has a tip taper 122 that is approximately 35 degrees and 0.5 inches in length, but the angle and length may be varied as long as comfort and ease of use is maintained. Balloon member 116 is, generally, resilient and pliable in construction, may be formed to have a smooth or textured finish, and may be made from materials such as medical grade rubbers, silicone, polyurethanes, or other suitable materials in any combination thereof, to permit inflation of balloon member 116, thereby expanding reservoir 120 as seen in FIGS. 5 and 7.

In FIG. 8A, a pelvic floor muscle or pelvic floor muscles 123 and a sphincter muscle or sphincter muscles 124 are at rest with a vaginal orifice 126, generally, being held closed by the resting, partially contracted, pelvic floor muscles 123 and sphincter muscles 124, respectively, known as the resting tone of a muscle. [Reference numerals 125-129 are in reference to other relevant female human anatomy to be explained further in FIGS. 8B-8D.] In FIG. 8B, balloon member 116, having a diameter smaller than that of vaginal orifice 126, is shown easily inserted into vagina 125 in such a manner that balloon member 116 extends beyond a vaginal pelvic floor muscle platform 127 and into a vaginal cavity 128. In FIG. 8C, a syringe 135 is connected to first end 112 by hose barb 136. Reservoir 120 is shown partially inflated with fluid or gas such as water or air, or any combination thereof, being received from syringe 135 through passageway 115. A clamp or clip 137 is selectively applied to tube 111 for controlling water or air flow through passageway 115. The inflated balloon member 116 engages a vaginal cavity edge 129 of vaginal cavity 128, thereby providing a weight resistance exercise as pelvic floor muscles 123 and sphincter muscles 124 are contracted and held for any proper period of time. In FIG. 8D, reservoir 120 is shown fully inflated with water or air, or any combination thereof, thereby providing a gradual weight resistance exercise as the diameter, length, and weight of balloon member 116 increases or decreases proportionally to the volume of water or air within reservoir 120. The fully inflated balloon member 116 can accommodate a high volume of water, or weight, and a low volume of air, or a low volume of water and a high volume of air, thereby fitting any size levator gap, referring to the relative gap between sphincter muscles 124 of vagina 125. A watertight lid or cap 138, commercially manufactured in the industry, is screwed onto hose barb 136, thereby retaining the contents of reservoir 120 and passageway 115.

In FIG. 9A, pelvic floor muscles 123 and sphincter muscles 124 are at rest with a rectal orifice 131, generally, being held closed by the resting, partially contracted, pelvic floor muscles 123 and sphincter muscles 124, respectively, known as the resting tone of a muscle. [Reference numerals 130-134 are in reference to other relevant human anatomy to be explained further in FIGS. 9B-9D.] In FIG. 9B, balloon member 116, having a diameter smaller than that of rectal orifice 131, is shown easily inserted into rectum 130 in such a manner that balloon member 116 extends beyond a rectal pelvic floor muscle platform 132 and into a rectal cavity 133. In FIG. 9C, syringe 135 is connected to first end 112 by hose barb 136. Reservoir 120 is shown partially inflated with water or air, or any combination thereof, being received from syringe 135 through passageway 115. Clip 137 is selectively applied to tube 111 for controlling water or air flow through passageway 115. The inflated balloon member 116 engages a rectal cavity edge 134 of rectal cavity 133, thereby providing a weight resistance exercise as pelvic floor muscles 123 and sphincter muscles 124 are contracted and held for any proper period of time. In FIG. 9D, reservoir 120 is shown fully inflated with water or air, or any combination thereof, thereby providing a gradual weight resistance exercise as the diameter, length, and weight of balloon member 116 increases or decreases proportionally to the volume of water or air within reservoir 120. The fully inflated balloon member 116 can accommodate a high volume of water, or weight, and a low volume of air, or a low volume of water and a high volume of air, thereby fitting any size levator gap, referring to the relative gap between sphincter muscles 124 of rectum 130. Cap 138 is screwed onto hose barb 136, thereby retaining the contents of reservoir 120 and passageway 115.

A second embodiment, generally indicated as exercise device 210, is configured in much the same manner as the preferred embodiment of FIGS. 1-7. In this embodiment, shown in FIGS. 10-14, a balloon member 216 covers a tube 211 from a second end 213 to a first end 212, having a seal 219 that extends from a reservoir 220 to first end 212.

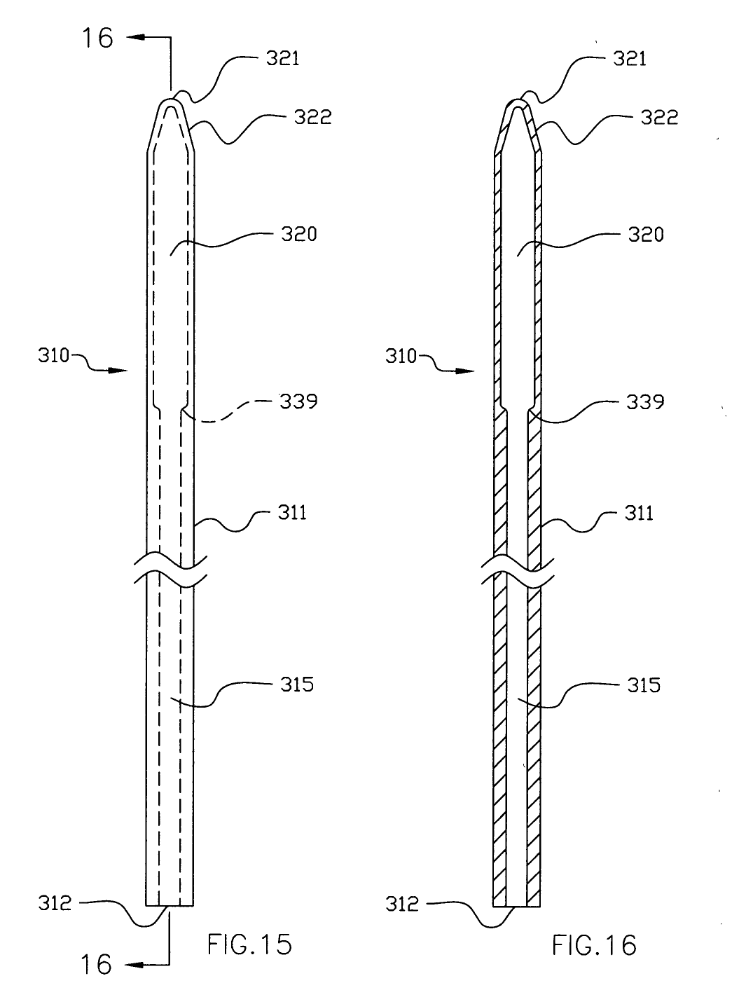

A third embodiment, generally indicated as exercise device 310, is configured in a similar manner as the second embodiment of FIGS. 10-14. In this embodiment, shown in FIGS. 15-19, a tube 311 is a single-layered hose that is approximately 0.375 inches in diameter, having a first end 312 and a tip end 321. Tip end 321 has a tip taper 322 that is similar to tip taper 122 in FIG. 1. An elongated passageway 315 that is approximately 0.125 inches in diameter, but the diameter may be varied as long as resistance to expansion is maintained, extends from first end 312 to a sharp-angled cut or reservoir edge 339. A reservoir 320 that is approximately 0.025 inches in diameter and 0.5 to 3.0 inches in length, but the diameter and length may be varied as long as resilient expansion is maintained, extends from reservoir edge 339 to tip end 321.

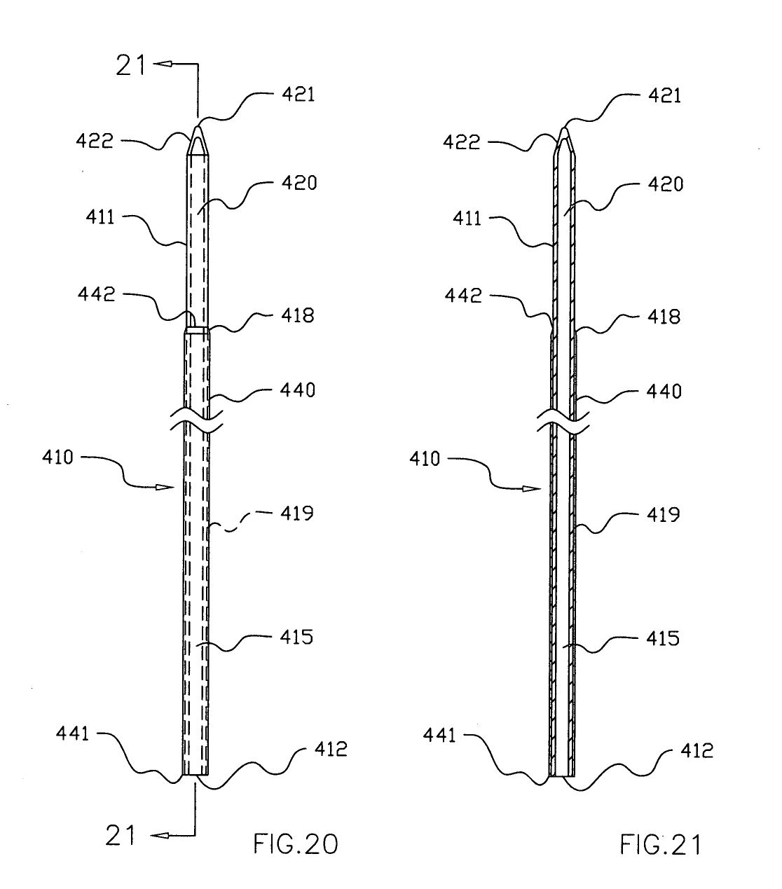

A fourth embodiment, generally indicated as exercise device 410, is configured in an inverse manner as the preferred embodiment of FIGS. 1-7. In this embodiment, shown in FIGS. 20-24, a tube 411 is an elastic hose or resilient tube having a first end 412 and a tip end 421. Tip end 421 has a tip taper 422 that is similar to tip taper 122 in FIG. 1. An elongated passageway 415 extends longitudinally through tube 411 from first end 412 to tip end 421. A non-expandable cover 440 is a semi-rigid hose having a first cover end 441 and a second cover end 442. Second cover end 442 has a bevel 418 around the outer edge. First end 412, having a diameter smaller than that of non-expandable cover 440, is inserted into non-expandable cover 440 from second cover end 442 to first cover end 441. Tube 411 is joined to non-expandable cover 440 by a seal 419 that extends from second cover end 442 to first cover end 441. Tube 411 extends beyond second cover end 442 approximately 0.5 to 3.0 inches, forming a pliable bag or reservoir 420.

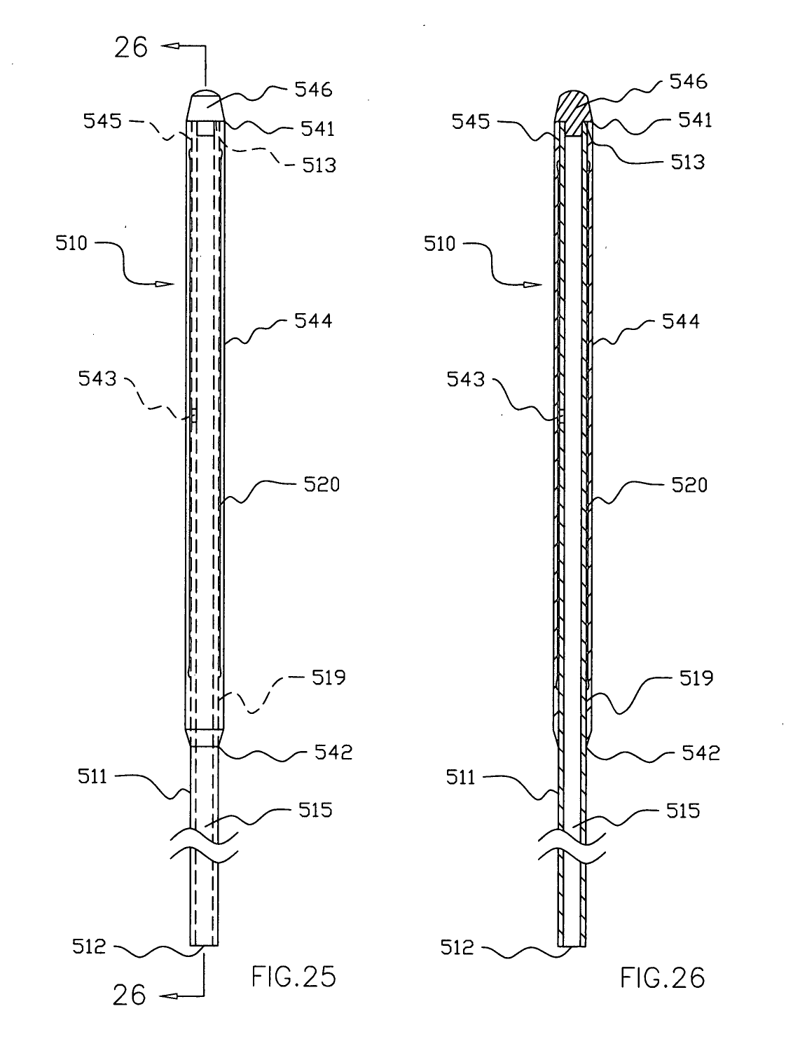

A fifth embodiment, generally indicated as exercise device 510, is configured in a similar manner as the preferred embodiment of FIGS. 1-7. In this embodiment, shown in FIGS. 25-29, a tube 511 has a first end 512 and a second end 513. A hole 543 that is approximately 0.09375 inches in diameter, but the diameter may be varied as long as ease of water or air flow is maintained, is cut into tube 511 approximately 1.0 to 3.0 inches from second end 513, thereby allowing water or air to flow through a passageway 515. A resilient tube or expandable cover 544 has a first cover end 541 and a second cover end 542 that is approximately 3.0 to 6.0 inches in length and 0.3125 inches in diameter, but the length and diameter may be varied as long as comfort and ease of use is maintained, that covers tube 511 from second end 513 to a seal 519 that is similar to seal 119 in FIG. 1. First cover end 541 is joined to second end 513 by a second seal 545 that is similar to seal 519, forming a reservoir 520. A tip-shaped cap or plug 546 is hermetically sealed to second seal 545, thereby closing first cover end 541 and second end 513.

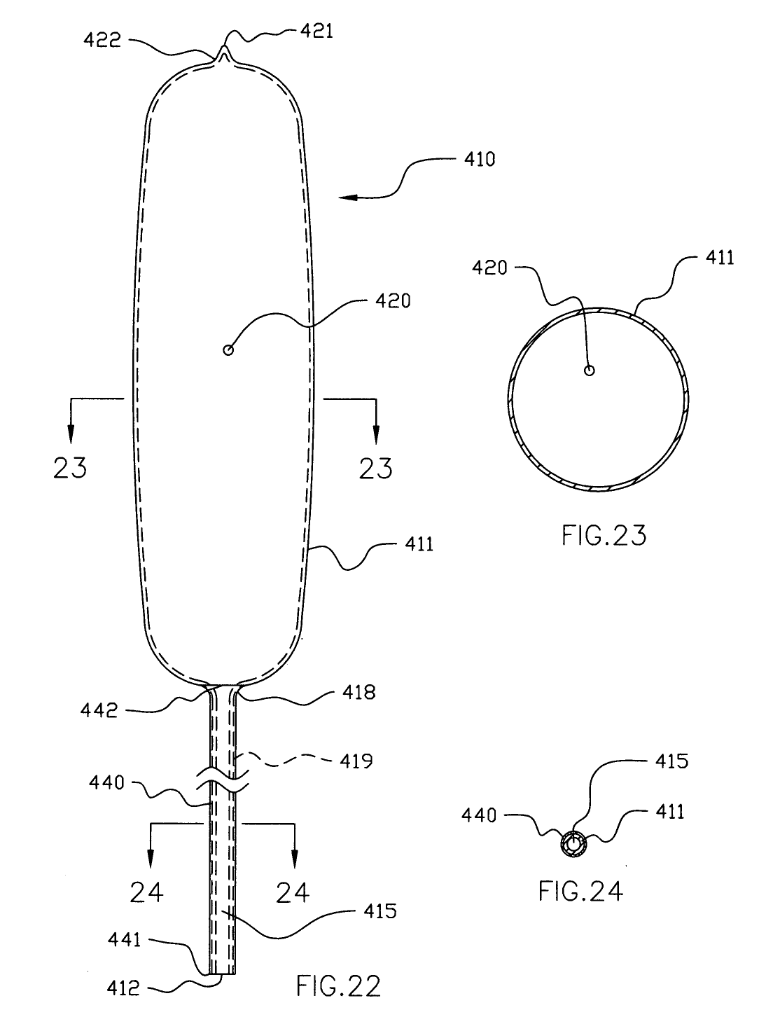

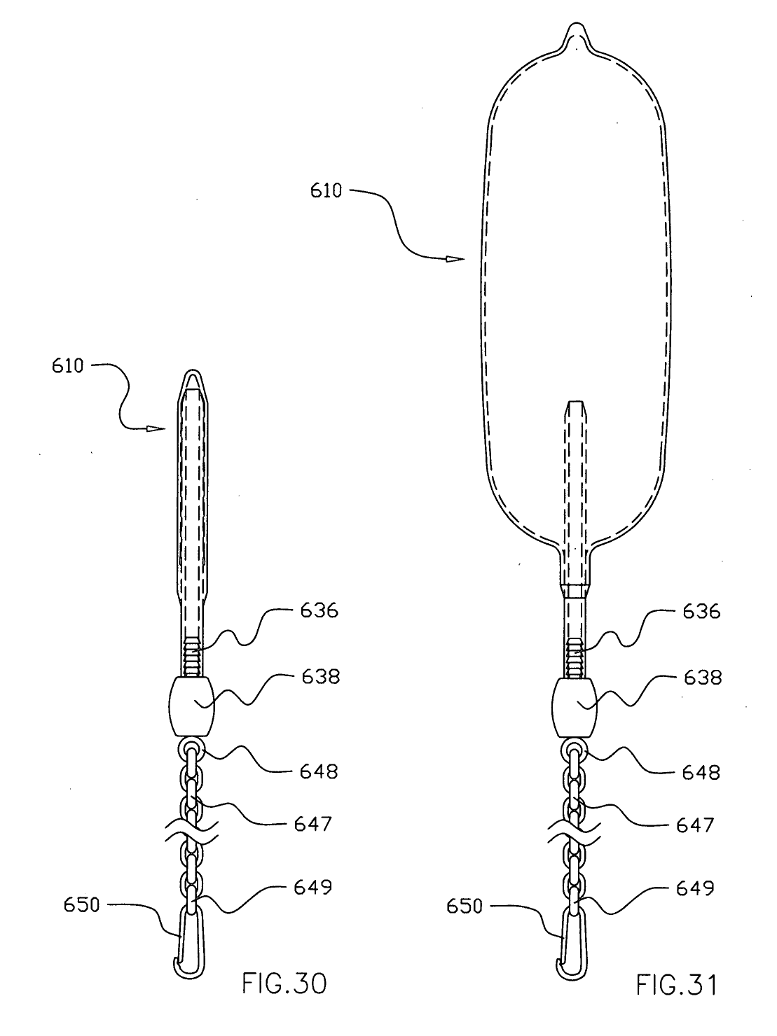

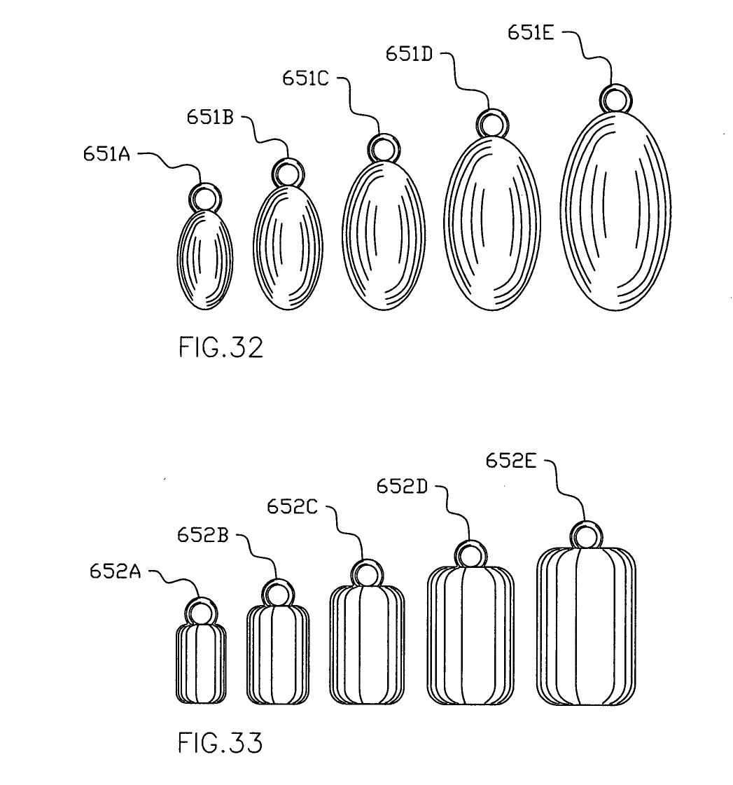

A sixth embodiment, generally indicated as exercise device 610, is configured in much the same manner as the preferred embodiment of FIGS. 1-7. In this embodiment, a cap 638, that is similar to cap 138 in FIG. 8D, is screwed onto a hose barb 636 as shown in FIGS. 30-33. A chain 647, commercially manufactured in the industry, that is approximately 1.0 to 6.0 inches in length, but the length may be varied as long as comfort and ease of use is maintained, has a first chain end 648 and a second chain end 649. First chain end 648 is permanently joined to the top of cap 638. A clasp hook 650, commercially manufactured in the industry, is connected to second chain end 649. A set of graduated weights, such as an oval-shaped graduated weight set 651A-651E in FIG. 32 or a barrel-shaped graduated weight set 652A-652E in FIG. 22, can be coupled to hook 650, thereby providing a wider variety of weight resistance training.

### Operation—FIGS. 8A-9D

Having thus set forth a preferred construction for the current invention, it is to be remembered that this is but the preferred embodiment. Attention is now invited to a description of the use of exercise device 110.

To use exercise device 110, the user should place clip 137 onto tube 111 as shown in FIGS. 8C and 9C. Close clip 137. Lubricate balloon member 116 with water or water-soluble lubricant. The user should squat with legs open and knees slightly bent or with one leg slightly elevated, sit, kneel, or lie down with knees up as long as the position is comfortable to the user. Insert deflated balloon member 116 into vagina 125 or rectum 130 until balloon member 116 extends beyond vaginal cavity edge 129 or rectal cavity edge 134 as shown in FIGS. 8B and 9B. The user may then sit or stand up using caution as to not kink tube 111. Using a faucet or container of water, fill syringe 135 with water or air, or any combination thereof. Holding syringe 135 in one hand and exercise device 110 in the other hand, connect syringe 135 to hose barb 136. Remove hand from exercise device 110 and open clip 137. Compress the plunger of syringe 135. Holding the plunger and syringe 135 with one hand, close clip 137 with the other hand. Remove syringe 135 from hose barb 136. If the desired volume has not been reached, refill syringe 135 and reconnect syringe 135 to hose barb 136. Holding the plunger and syringe 135 with one hand, remove other hand from exercise device 110 and open clip 137 using caution as to hold plunger firmly against pressure produced by balloon member 116. Compress the plunger of syringe 135. Holding the plunger and syringe 135 with one hand, close clip 137 with the other hand. Remove syringe 135 from hose barb 136. Continue filling reservoir 120 until desired diameter, length, and weight has been reached. Screw cap 138 onto hose barb 136 as shown in FIGS. 8D and 9D. Remove clip 137 from tube 111.

When used as an exercise device, the user should insert the deflated balloon member 116 into vagina 125 or rectum 130 and fill with a combination of fluid or gas such as water or air to find a comfortable working size and weight in which the user can hold exercise device 110 inside vaginal cavity 128 or rectal cavity 133 without slipping out. The user must hold the exercise device 110 by contracting the pelvic floor muscles 123 and sphincter muscles 124 for approximately 10 to 60 minutes once or twice a day for 4 to 8 weeks. The user should gradually increase the weight of the exercise device 110 as pelvic floor muscles 123 and sphincter muscles 124 as shown in FIGS. 8A and 9A, are sufficiently strengthened. After that, the user might exercise periodically to maintain muscle strength. The user might keep a written record of their exercise progress, which provides additional feedback to the user as the user can readily ascertain their progress over a period of time and see their improvement.

When used as a test device, the user would keep increasing the weight of exercise device 110 until it could no longer be retained in vagina 125 or rectum 130. This provides an incremental measurement of the relative strength of the pelvic floor muscles 123 and sphincter muscles 124.

To empty exercise device 110, the user should place clip 137 onto tube 111 as shown in FIGS. 8C and 9C. Close clip 137. Pointing cap 138 towards a drain or container, unscrew cap 138 from hose barb 136. Open clip 137 allowing exercise device 110 to drain completely. Remove exercise device 110 from vagina 125 or rectum 130. Remove clip 137 from tube 111. Wash outside and inside of exercise device 110 with soap and water, alcohol, or peroxide.

### CONCLUSION, RAMIFICATIONS, AND SCOPE

Accordingly, the reader will see that the exercise device of this invention can be easily used to test and strengthen the muscle mass, strength, and tone of the pelvic diaphragm. Furthermore, the exercise device has the additional advantages in that
a) it permits the user to correct medical problems without submitting to the dangers of surgery or negative side effects from medications;
b) it provides a painless insertion and removal process due to the small diameter of the deflated exercise device without further damaging muscles of the pelvic diaphragm;
c) it allows the user to adjust the diameter, length, and weight of the exercise device to accommodate any size levator gap and pelvic diaphragm muscle strength;
d) it permits the appropriate muscles to be exercised without the assistance of a user's hands or a doctor;
e) it allows the user to discreetly exercise in private or public places for any proper period of time;
f) it is portable and can be adjusted anywhere there is water;
g) it provides superior comfort and flexibility by conforming to the natural structure of each user;
h) it remains more securely in place due to its cylindrical shape;
i) it provides a simple, and easy way to inflate, deflate, and clean; and
j) it can be manufactured and sold at a low cost.

Although the description above contains many specifications of our exercise device, these should not be construed as limiting the scope of the invention, but as merely providing illustrations of some of the presently preferred embodiments of this invention. For example, the exercise device can have other shapes, such as circular, oval, oblong, elliptical, conical, etc.; the materials can be of different colors such as tan, pink, white, etc.; the materials can be of different finishes, such as smooth, textured, etc.; the materials can be of varying elasticity; the clip and hose barb can be a single manufactured part.

Thus, the scope of the invention should be determined not by the embodiments illustrated, but by the appended claims and their legal equivalents.

We claim:

1. An exercise device for testing and/or strengthening muscles of the pelvic diaphragm, comprising:
a. an elongated semi-rigid tube having a first end and a second end;
b. said tube having a passageway extending longitudinally from said first end to said second end;
c. a resilient balloon member surrounding said second end of said tube and sealed thereto forming a reservoir; and
d. said reservoir being of such dimensions that, when a predetermined fluid or gas pressure is developed, it expands to form a shape of substantially uniform cross-sectional dimensions.

2. An exercise device as in claim 1, wherein said tube has an outer diameter that is approximately 0.25 inches and an inner diameter that is approximately 0.125 inches.

3. An exercise device as in claim 1, wherein said second end has a tube taper that is approximately 35 degrees.

4. An exercise device as in claim 1, wherein said balloon member has an outer diameter that is approximately 0.3125 inches and an inner diameter that is approximately 0.25 inches, whereby insertion into the vagina or rectum can be performed without additional damage to muscles of the pelvic diaphragm.

5. An exercise device as in claim 1, wherein said balloon member has a cylindrical shape.

6. An exercise device as in claim 1, wherein said balloon member has a circular shape.

7. An exercise device as in claim 1, wherein said balloon member has a conical shape.

8. An exercise device as in claim 1, wherein said exercise device is composed of rubber.

9. An exercise device as in claim 1, wherein said exercise device is composed of silicone.

10. An exercise device as in claim 1, wherein said exercise device is composed of polyurethanes.

11. An exercise device as in claim 1, wherein said exercise device has a smooth finish.

12. An exercise device as in claim 1, wherein said exercise device has a textured finish.

13. An exercise device as in claim 1, further comprising a means of connecting a syringe or other plunging device to said exercise device, whereby said syringe is detachable from the connecting means.

14. An exercise device as in claim 1, further comprising a plurality of weights that are made of the same material but have different sizes so as to have different weight, whereby the range of exercise weight is increased.

15. An exercise device as in claim 14, further comprising a means of connecting said plurality of weights to said exercise device, whereby said plurality of weights are detachable from the connecting means.

16. An exercise device for testing and/or strengthening muscles of the pelvic diaphragm, comprising:
a. an elongated resilient tube having a first end and a tip end;
b. said tube having a passageway of varied inner diameter extending longitudinally from said first end to said tip end;
c. said passageway having a substantially larger inner diameter extending approximately one-half to three inches from said tip end forming a reservoir; and
d. said reservoir being of such dimensions that, when a predetermined fluid or gas pressure is developed, it expands to form a shape of substantially uniform cross-sectional dimensions.

17. An exercise device for testing and/or strengthening muscles of the pelvic diaphragm, comprising:
a. an elongated resilient tube having a first end and a tip end;
b. said tube having a passageway extending longitudinally from said first end to said tip end;
c. a semi-rigid non-expandable cover surrounding said first end of said tube and sealed thereto such that approximately 0.5 to 3.0 inches of said tip end of said tube extends beyond said non-expandable cover forming a reservoir; and
d. said reservoir being of such dimensions that, when a predetermined fluid or gas pressure is developed, it expands to form a shape of substantially uniform cross-sectional dimensions.

18. An exercise device for testing and/or strengthening muscles of the pelvic diaphragm, comprising:
a. an elongated semi-rigid tube having a first end and a second end;
b. said tube having a passageway extending longitudinally from said first end to said second end;
c. said tube having a hole that is approximately 1.0 to 3.0 inches from said second end;
d. a tubular-shaped expandable cover having a first cover end and a second cover end surrounding said second end of said tube;
e. said expandable cover being sealed to said tube at said first cover end and said second cover end forming a reservoir;
f. a tip-shaped plug being sealed to said first cover end of said expandable cover and said second end of said tube such that said first cover end and said second end are closed; and
g. said reservoir being of such dimensions that, when a predetermined fluid or gas pressure is developed, it expands to form a shape of substantially uniform cross-sectional dimensions.

19. An exercise device as in claim 18, wherein said tube has a plurality of holes.

20. A method of testing and/or strengthening muscles of the pelvic diaphragm, comprising the steps of:
a. gripping a second end of an exercise device;
b. inserting said second end of said exercise device into a vagina or a rectum such that a balloon member is disposed within a vaginal cavity or a rectal cavity and a first end of said exercise device is protruding from said vagina or said rectum;
c. filling said exercise device with fluid such as water such that said balloon member expands increasing in diameter, length, and weight;
d. checking whether the muscles of the pelvic diaphragm can support the weight of said exercise device;
e. if so, adding additional fluid, and if not, removing some fluid until said exercise device can be held by voluntary holding, the weight of said exercise device providing an indication of the pelvic diaphragm muscle strength;
f. filling said exercise device with gas such as air such that said balloon member expands increasing in diameter and length;
g. checking whether the levator gap can support the diameter of said exercise device;
h. if so, removing some gas, if not, adding additional gas until said exercise device can be held by voluntary holding, the diameter of said exercise device providing an indication of the size of the levator gap;
i. retaining said exercise device by exerting voluntary holding for a predetermined time; and
j. removing said exercise device from said vagina or said rectum;
whereby said exercise device can increase the muscle mass, strength, and tone of the pelvic diaphragm muscle structure to correct medical problems such as incontinence without submitting to the dangers of surgery or negative side effects from medications.
